(19) United States
(12) Patent Application Publication
(10) Pub. No.: US 2005/0149163 A1
(43) Pub. Date: Jul. 7, 2005

(54) Reduced restenosis drug containing stents

(76) Inventor: Harvinder Sahota, Seal Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/057,991

(22) Filed: Feb. 15, 2005

Related U.S. Application Data
(62) Division of application No. 10/103,409, filed on Mar. 20, 2002.

(51) Int. Cl. A61F 2/06

(52) U.S. Cl. 623/1.11

(57) ABSTRACT

A drug delivery stent and stent delivery system and method are provided. The stent comprises at least two therapeutic agents. In one embodiment, at least two therapeutic agents are administered at dosage levels that a lower than conventional dosing, in order to reduce the risk of side-effects. In another embodiment, the first agent is preferably a slow-release agent, while the second agent is a quick-release agent. These agents are administered using a stent.

### RELATED APPLICATIONS

This application is a divisional of application Ser. No. 10/103,409, filed Mar. 20, 2002.

### BACKGROUND OF THE INVENTION

1. Field of the Invention

The present invention relates to medical devices, and in particular to drug delivery stents.

2. Description of the Related Art

Many diseases cause body lumens to undergo stenosis or a narrowing of a canal within the body. The resulting shortage of blood flow can permanently damage tissue and organs. Stenotic regions that limit or obstruct coronary blood flow are the major cause of ischemic heart disease related mortality and result in 500,000-600,000 deaths in the United States annually.

The therapeutic alternatives available for treatment of stenosis include intervention (alone or in combination of therapeutic agents) to remove the blockage, replacement of the blocked segment with a new segment of artery, or the use of a catheter-mounted device such as a balloon catheter to dilate the artery. The dilation of an artery with a balloon catheter is called percutaneous transluminal angioplasty (PTA). During angioplasty, a balloon catheter in a deflated state is inserted within a stenotic segment of a blood vessel and is inflated and deflated a number of times to expand the vessel.

Often angioplasty permanently opens previously occluded blood vessels; however, restenosis, thrombosis, or vessel collapse may occur following angioplasty. A major difficulty with PTA is the problem of post-angioplasty closure of the vessel, both immediately after PTA (acute reocclusion) and in the long term (restenosis). Recently, intravascular stents have been examined as a means of preventing acute reclosure after PTA.

Restenosis refers to the re-narrowing of an artery after an initially successful angioplasty due to exaggerated healing which causes a proliferation of tissue in the angioplasty area. Thrombosis is a clotting within a blood vessel which may cause infarction of tissues supplied by the blood vessel.

Re-narrowing (restenosis) of an artery after angioplasty occurs in 10-50% of patients undergoing this procedure and subsequently requires either further angioplasty or more invasive surgical procedures. While the exact processes promoting restenosis are still under investigation, the process of PTA is believed to injure resident arterial smooth muscle cells (SMC). In response to this injury, adhering platelets, infiltrating macrophages, leukocytes, or the smooth muscle cells (SMC) themselves release cell derived growth factors. Many other potential reasons are also being investigated.

Restenosis (chronic reclosure) after angioplasty is a more gradual process than acute reocclusion: 30% of patients with subtotal lesions and 50% of patients with chronic total lesions will go on to restenosis after angioplasty.

Because 30-50% of patients undergoing PTCA will experience restenosis, restenosis has clearly limited the success of PTCA as a therapeutic approach to coronary artery disease. Because SMC proliferation and migration are intimately involved with the pathophysiological response to arterial injury, prevention of SMC proliferation and migration represents a target for pharmacological intervention in the prevention of restenosis.

In order to prevent restenosis and vessel collapse, stents of various configurations have been used to hold the lumen of a blood vessel open following angioplasty.

Most stents are compressible for insertion through small cavities, and are delivered to the desired implantation site percutaneously via a catheter or similar transluminal device. Once at the treatment site, the compressed stent is expanded to fit within or expand the lumen of the passageway. Stents are typically either self-expanding or are expanded by inflating a balloon that is positioned inside the compressed stent at the end of the catheter. Intravascular stents are often deployed after coronary angioplasty procedures to reduce complications, such as the collapse of arterial lining, associated with the procedure.

However, stents do not entirely reduce the occurrence of thrombotic abrupt closure due to clotting; stents with rough surfaces exposed to blood flow may actually increase thrombosis, and restenosis may still occur because tissue may grow through and around the lattice of the stent.

In addition to providing physical support to passageways, stents are also used to carry therapeutic substances for local delivery of the substances to the damaged vasculature. For example, anticoagulants, antiplatelets, and cytostatic agents are substances commonly delivered from stents and are used to prevent thrombosis of the coronary lumen, to inhibit development of restenosis, and to reduce post-angioplasty proliferation of the vascular tissue, respectively. The therapeutic substances are typically either impregnated into the stent or carried in a polymer that coats the stent. The therapeutic substances are released from the stent or polymer once it has been implanted in the vessel.

Several recent experimental approaches to preventing SMC proliferation have shown promise although the mechanisms for most agents employed are still unclear. Heparin is a well known and characterized agent causing inhibition of SMC proliferation.

Other agents which have demonstrated the ability to reduce myointimal thickening in animal models of balloon vascular injury are angiopeptin (a somatostatin analog), calcium channel blockers, angiotensin converting enzyme inhibitors (captopril, cilazapril), cyclosporin A, trapidil (an antianginal, antiplatelet agent), terbinafine (antifungal), colchicine and taxol (antitubulin antiproliferatives), and c-myc and c-myb antinsense oligonucleotides.

Additionally, a goat antibody to the SMC mitogen platelet derived growth factor (PDGF) has been shown to be effective in reducing myointimal thickening in a rat model of balloon angioplasty injury, thereby implicating PDGF directly in the etiology of restenosis. Thus, while no therapy has as yet proven successful clinically in preventing restenosis after angioplasty, the in vivo experimental success of several agents known to inhibit SMC growth suggests that these agents as a class have the capacity to prevent clinical restenosis and deserve careful evaluation in humans.

However, a number of side effects have been associated with current usage of these drugs. A stent to improve upon the existing problems that can deliver multiple drugs at lower doses is described herein.

### SUMMARY OF THE INVENTION

Although research has concentrated on trying to show a particular cause of restenosis, restenosis appears to stem from multiple causes. A drug-delivery stent and stent delivery system disclosed in this application system delivers at least two, and even multiple drugs to a treatment site. Thus, treatment for different causes may be administered with a combination of drugs. In addition, more than one drug may be used for the same cause of restenosis, such that a reduced dosage may be administered, with lower risk of side-effects, and/or a more effective treatment of the cause. In addition, more than one drug may be administered for multiple causes of restenosis. In one embodiment, both long term acting and short term acting agents are utilized. The present invention also includes a method for delivering such drugs to a treatment site. A stent may include balloon-expanding stents, self-expanding stents, or tubular graft stents.

In one embodiment, a drug delivery stent has a stent structure configured to carry at least two therapeutic agents. At least a first therapeutic agent is provided in low dosage, and at least a second therapeutic agent is provided in low dosage, wherein the dosage levels of the at least first and second therapeutic agents are selected to reduce the risk of side effects compared to either agent administered alone at a standard dosing. Preferably, at least one of the first and second therapeutic agents is administered via the stent in a slow release manner. In one embodiment, at least one of the agents is administered in a quick release manner or is a quick release agent.

In another embodiment, a drug-delivery stent has at least a first drug, wherein the first drug is a quick-release drug, and at least a second drug, wherein the second drug is a slow-release drug.

The agents or drugs are typically selected from a group consisting of heparin, heparin derivatives, heparin fragments, colchicine, angiopeptin, steroids, gene vectors, cortisone, taxol, nitric oxide, carbide, docetaxel, mthotrexate, azathiprine, vincristine, vinblastine, fluorouracil, doxorubicin hydrochloride, mitomycin, heparinoids, hirudin, argatroban, forskolin, vapiprost, prostacyclin, protacyclin analogues, dextran, dipryidamole, recombinant hirudin, captrpril, cilazapril, lisinopril, calcium channel blockers, fish oil, histamine antagonists, lovastatin, dipryidamole, monoclonal antibodies, suramin, seratonin blockers, thioprotease inhibitors, triazolpyrimidine, permirolast potassium, dexamethason, radioactive isotopes, phosphoric acid, palladium, cesium, iodine and aspirin.

The agents may also be selected from the group consisting of anti-thrombotics, anti-inflammatories, anti-proliferatives, antineoplastic, antiplatelet, antifibrin, antibiotic, antioxidant, anti-allergic drugs, angiogenic drugs, smooth muscle cell inhibitors, anti-coagulents, cholesterol reducing agents, calcium antagonists, thromboxane inhibitors, prostacyclin mimetics, platelet membrane receptor blockers, thrombin inhibitors, angiotensin converting enzyme inhibitors and combinations thereof. Naturally, any agent with therapeutic benefits in the context of a stent may be utitlized.

In one embodiment, third, fourth and even additional agents may be administered via the stent.

The stent structure may take many forms, such as balloon-expandable stent device, a self-expanding stent device, a tubular graft stent device and any other type of stent structure. These may be constructed in many ways such as helices, coils, braids, expandable tube stents, roving wire, and wire mesh. The drugs may be contained within pits, pores, grooves, reservoirs, or protruding structures having central depressions or combinations thereof, or any other structure or part of the stent that can contain the agents. The agents may also be a coating or thin film.

A method for treating a stenosed body lumen is also disclosed, which involves delivering a stent to the body lumen, delivering at least two drugs to the patient via the stent. In one embodiment, the at least two drugs comprise a first quick-release drug and a second slow-release drug. In one embodiment, the at least two therapeutic agents or drugs are administered at a dosage level that is low enough such that the risk of side effects from the combination of therapeutic agents is reduced in comparison to the administration of the same agents at conventional dosages.

In one embodiment, the method first involves testing the patient for allergies, and delivering therapeutic agents to which the patient is not allergic.

### BRIEF DESCRIPTION OF THE DRAWINGS

FIG. 1 is a perspective view showing a catheter having a stent of the present invention.

FIG. 2 is a cross-sectional view showing the catheter of FIG. 1 through line 2-2.

FIG. 3 is a detailed cross-sectional view of the distal end of the catheter and stent of FIG. 1 through line 3-3.

FIG. 4 is a perspective view showing an alternative embodiment of a catheter having a stent of the present invention.

FIG. 5 is a cross-sectional view showing the catheter of FIG. 4 through line 5-5.

FIG. 6 is a detailed cross-sectional view of the distal end of the catheter and stent of FIG. 4 through line 6-6.

FIGS. 7A-7H are detailed views of stents of the present invention.

FIG. 71 is a detailed cross-sectional view of a stent of the present invention.

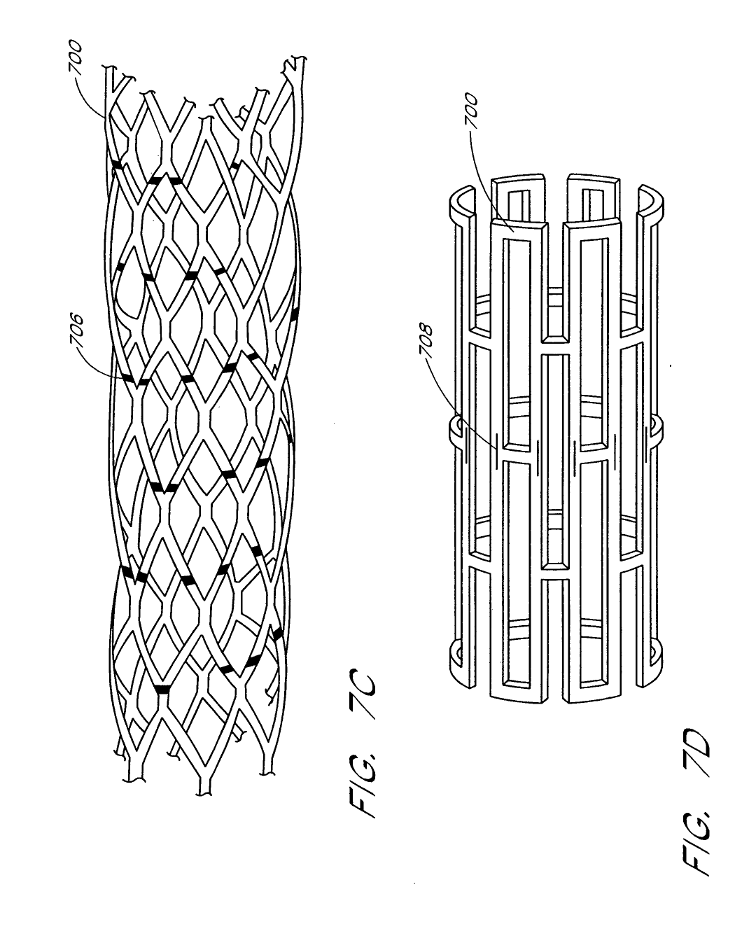

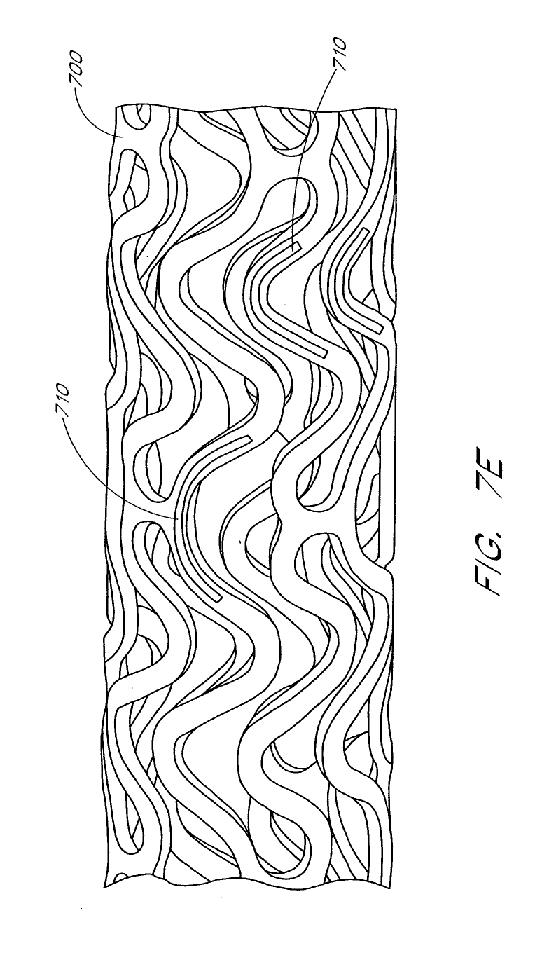

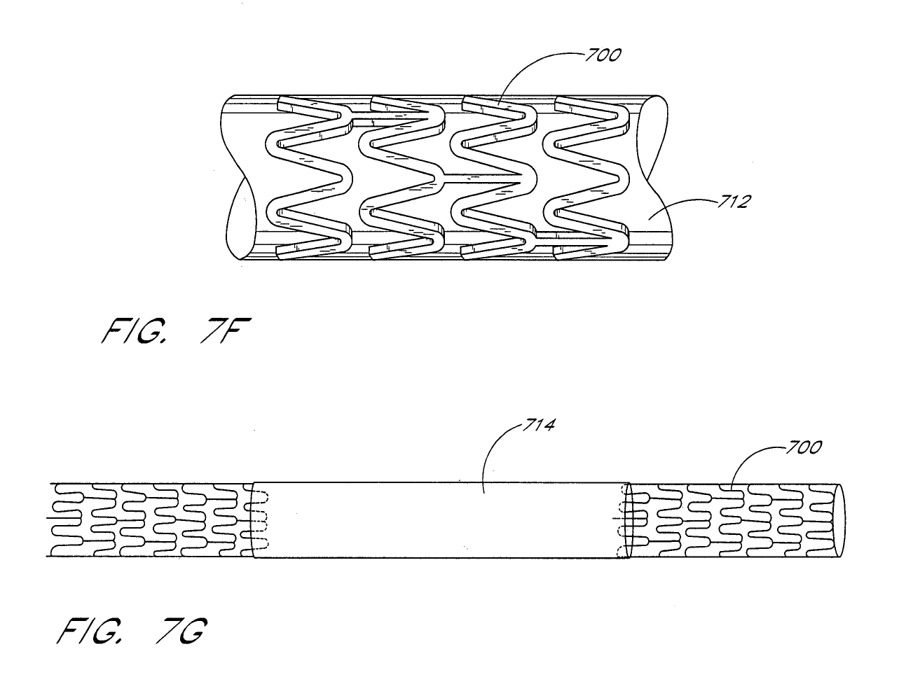

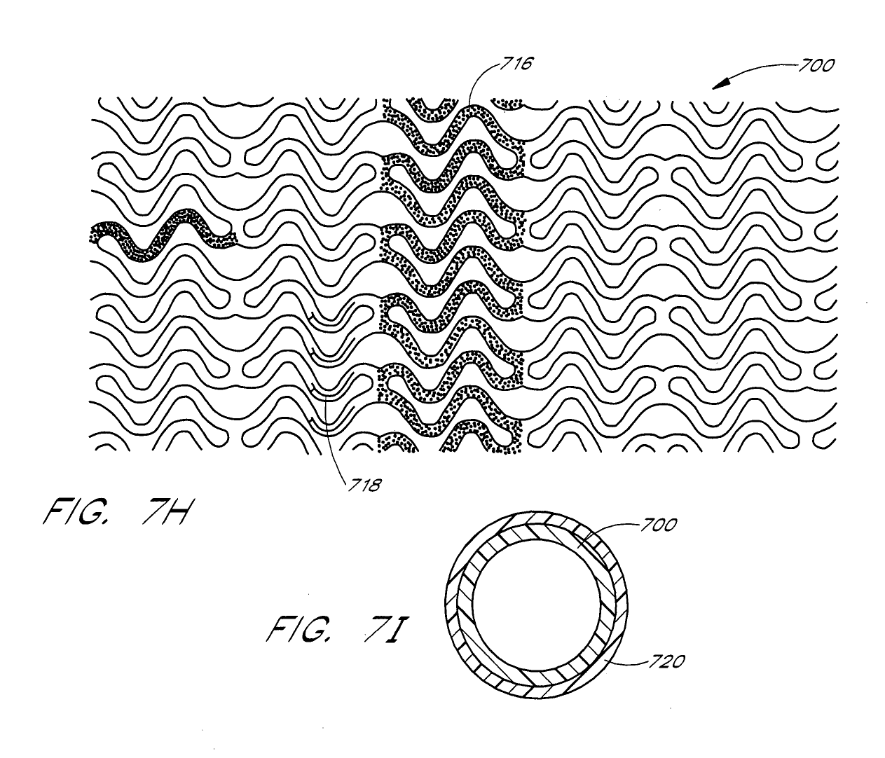

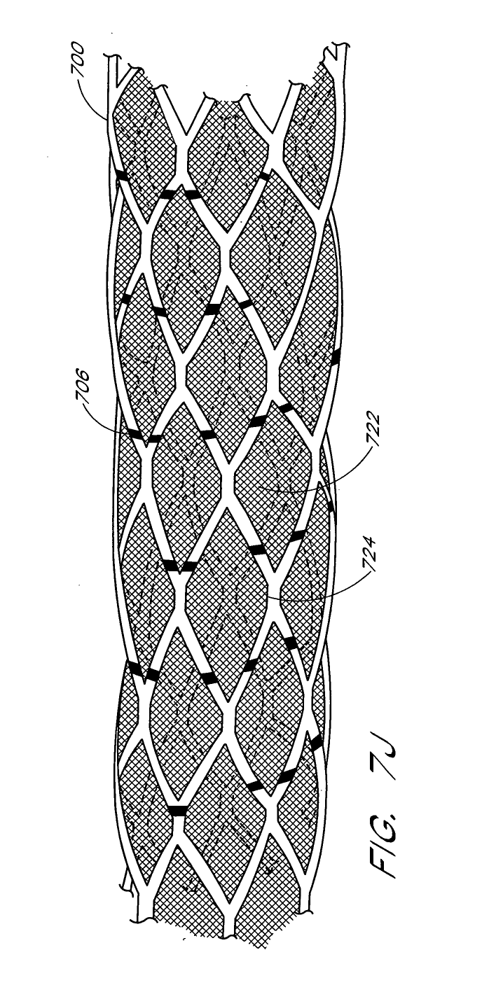

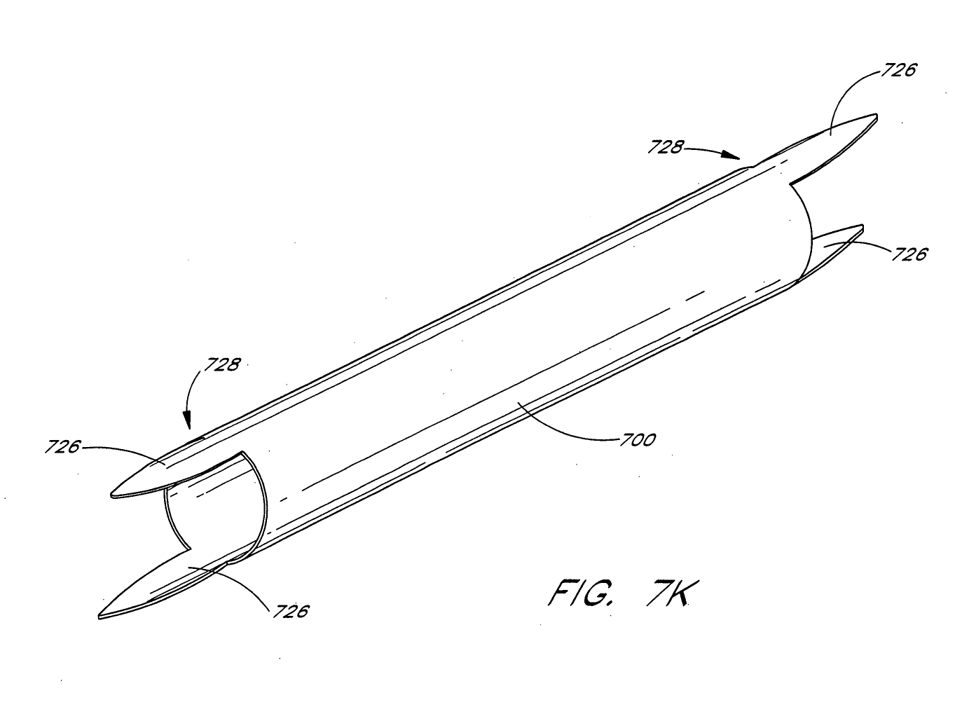

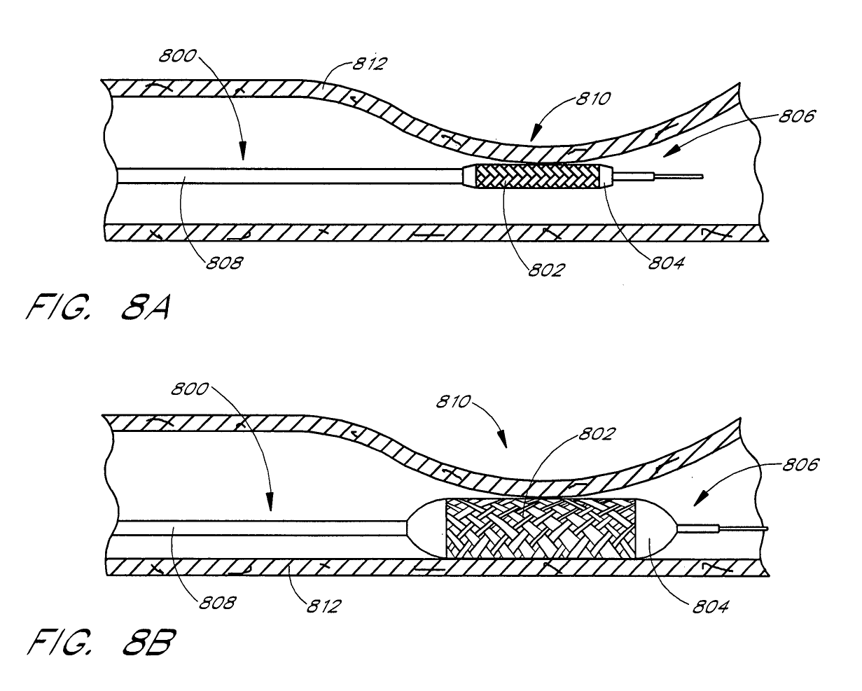

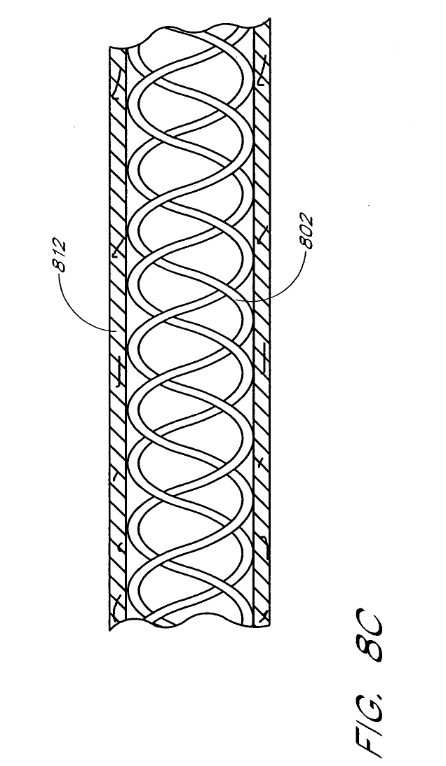

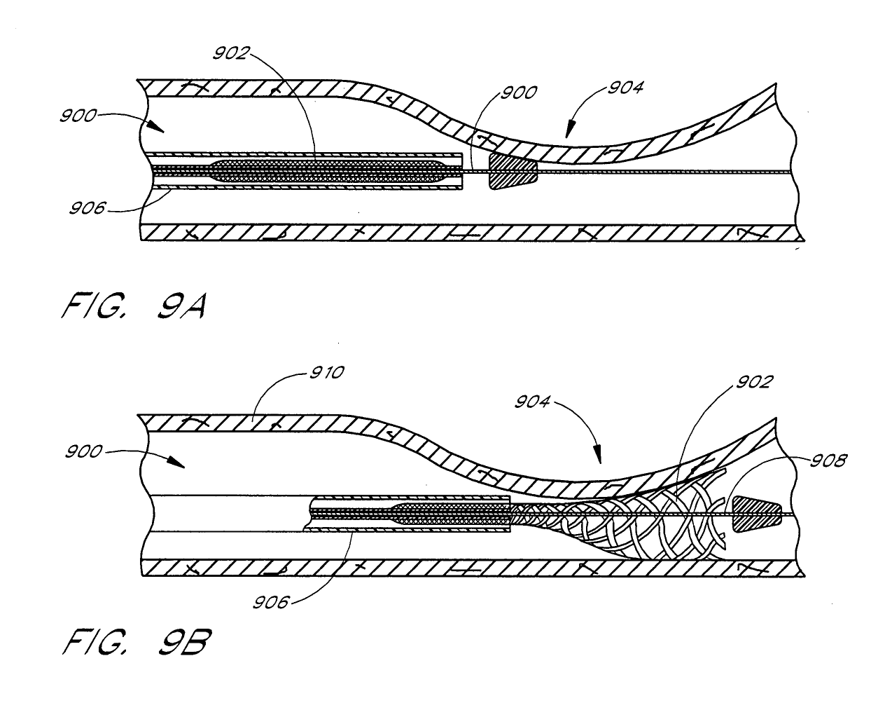

FIG. 7J is a detailed view of a stent of the present invention.

FIG. 7K is a detailed view of an alternative embodiment of a stent of the present invention.

FIGS. 8A-C are sectional views of a stenosed vessel showing the method of the present invention.

FIGS. 9A-C are sectional views of a stenosed vessel showing the method of the present invention.

### DETAILED DESCRIPTION OF THE PREFERRED EMBODIMENT

The following detailed description presents various specific embodiments of the present invention. However, such embodiments are illustrative of the invention and do not restrict the invention. A multitude of different forms are possible, and the invention is defined by the claims with the claim terms used in their ordinary and customary meaning. In this description, reference is made to the drawings wherein like parts are designated with like numerals.

A stent delivery catheter system in which a stent is delivered intraluminally into a body lumen, such as a coronary artery, carotid artery, renal arteries, peripheral arteries and veins, and the like is disclosed. The catheter system is also useful in the brain and the urethral system. The present invention comprises an improved drug delivery stent and method of delivering a therapeutic agent to a patient.

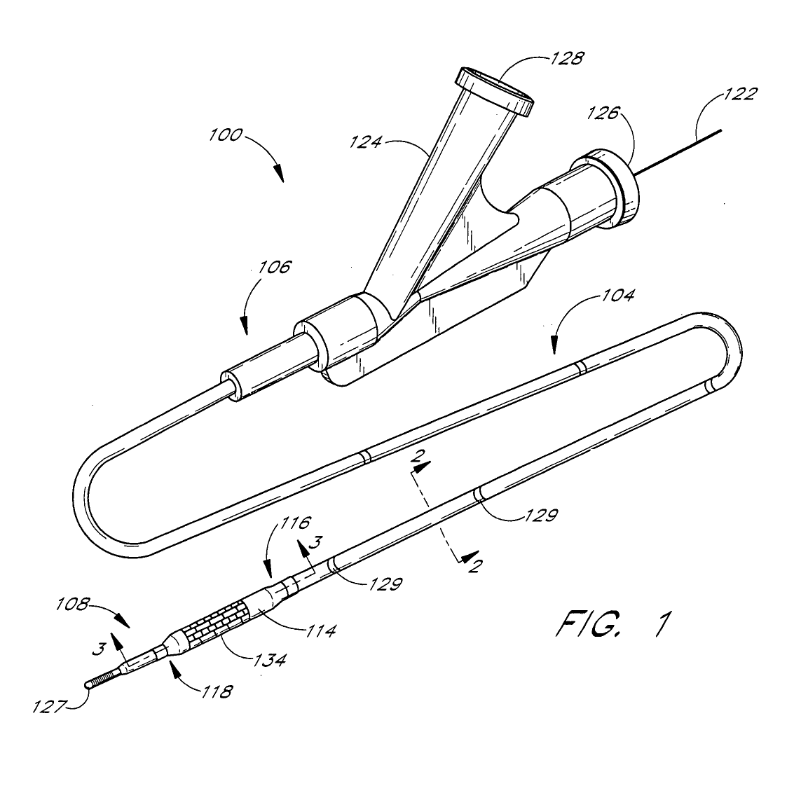

With reference to FIG. 1, a stent delivery catheter 100 is shown. Delivery catheter 100 preferably includes an elongate, flexible tubular shaft 104, having a proximal end 106 and a distal end 108. The shaft 104 defines one or more passages or lumens extending through the shaft.

Catheter 100 preferably comprises a balloon 114, having a proximal end 116 and a distal end 118. Elongate shaft 104 preferably includes a guide wire 122, extending from distal end 116 through proximal end 106 of shaft 104, providing rigidity to device 100. Catheter 100 also includes a manifold 124. Manifold 124 preferably includes a guide wire port 126 and an inflation port 128. Catheter 100 may also include radiopaque markers 129 to view the location of catheter 100 within the patient's body lumen. Catheter 100 may also include a soft, flexible distal tip 127. Such catheters are know.

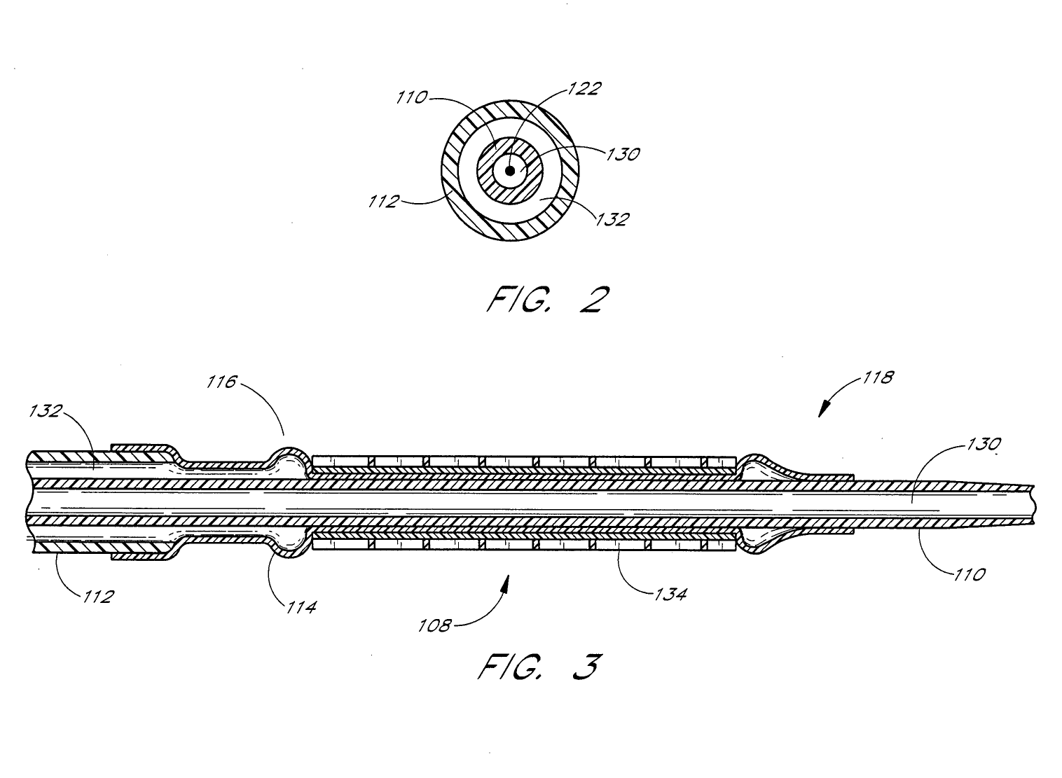

FIG. 2 shows a cross-sectional view of the disclosed embodiment of the elongate shaft 104, showing inner sleeve 110 and outer sleeve 112. The inner sleeve 110 defines a guide wire lumen 130, while the inflation lumen 132 is defined by the annular space between the inner sleeve 110 and outer sleeve 112. The guide wire lumen 130 is adapted to receive an elongate guide wire 122 in a sliding fashion through proximal guide wire port 126 in catheter manifold 124.

Preferably, inflation lumen 132 is connected to the balloon 114 to selectively inflate it with the inflating fluid. The inflation lumen 132 provides fluid communication between the interior of the balloon 114 at the distal end of the inflation lumen 132 and the inflation port 128 located at manifold 124.

The inflation lumen 132 may also be adapted to hook up to a vacuum, to eliminate air bubbles. Alternatively, a separate lumen may be provided for connection with the vacuum. Vacuum lumen would also be in communication with the internal cavity of balloon 114.

The catheter shaft 104 may have various configurations other than the coaxial design shown in the drawings, including a single extruded multi-lumen tube defining any suitable number of colinear or radially aligned lumens.

Stent 134 is preferably removably carried by the distal end 108 of elongate shaft 104. Stent 134 has an initial diameter at which it is inserted into a body lumen, and an expanded final diameter. Stent 134, as shown in FIGS. 1 and 3, is a balloon-expandable slotted metal tube (usually but not limited to stainless steel), which when expanded within the lumen, provides structural support to the arterial wall. Stent 134 comprises a tubular structure, having an inner lumen 136. Although stent 134 is illustratively shown in the configuration 100 of FIG. 1, the stent 100 may be of virtually any configuration so long as stent 100 meets the needs of the treatment procedures. Configurations, such as helices, coils, braids, expandable tube stents, roving wire stents, and wire mesh stents or the like may be utilized depending on the application for the device.

The balloon 114 may comprise a substantially inelastic, compliant material. Many balloon configurations are known. The balloon 114 is formed from any suitable material, but preferably from a biocompatible-braided polymer, such as polyamide, polyethylene or polyurethane. Other suitable materials include Nylon, PEEK, Pebax, or a block copolymer thereof.

The balloon 114 is preferably removably attached to the catheter shaft 104 by affixing its distal end to the inner sleeve 110, and its proximal end to the outer sleeve 112. The balloon 114 thereby communicates with the annular inflation lumen 132 between the inner sleeve 110 and outer sleeve 112. The balloon 114 may alternatively be attached to the shaft 104 in any way that allows it to be inflated with fluid from the inflation lumen 132.

The catheter manifold 124 provides a maneuvering handle for the health care professional, as well as an inflation port 128 and a guide wire port 126. Either or both the inflation port 128 or the guide wire port 126 may have a coupling, accompanied by a luer-lock fitting for connecting an inflation lumen to a source of pressurized fluid in a conventional manner. The manifold 124 may also include an injection port for allowing radiopaque contrast fluid to be injected through the outer sleeve and around the catheter shaft, thus illuminating the delivery device on a fluoroscope. The proximal manifold 124 is preferably injection molded of any suitable material. A precision gasket may also be provided, which seals securely around the device, prohibiting fluid loss. Many other catheter configurations are also known.

The size of stent 134 varies, depending on the particular treatment and access site. The overall length, diameter and wall thickness may vary based on the treatment. In a preferred embodiment, stent 134 has an inflated length between about 1 and 10 cm, preferably about 4 cm. In a preferred embodiment, stent 134 has an inflated diameter between about 0.1 and 1.5 cm. However, stents of any dimensions may be used.

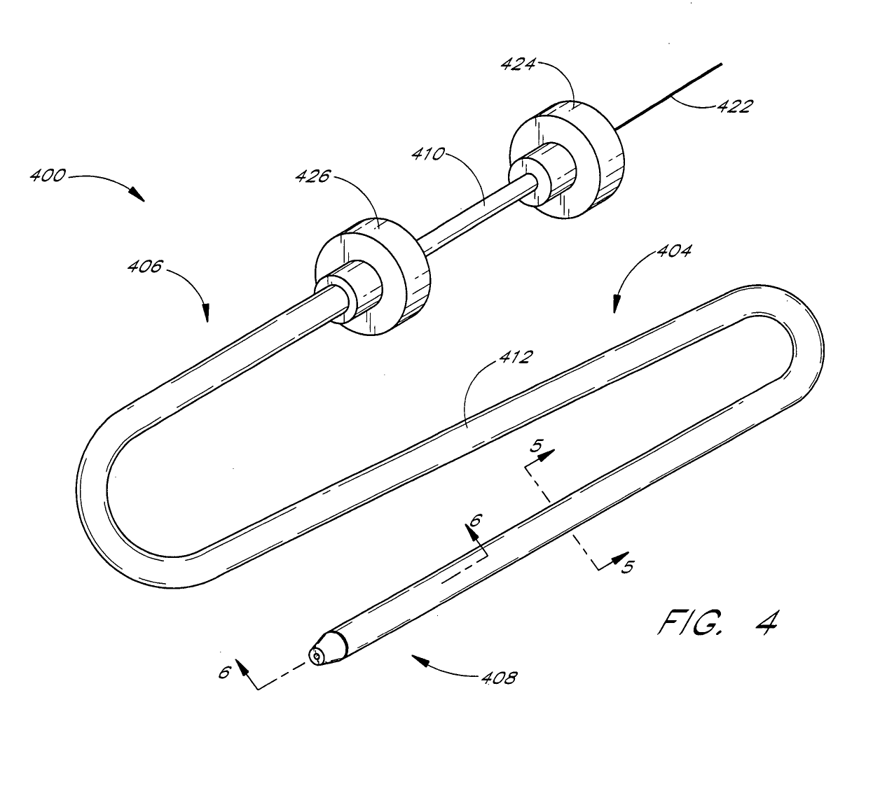

With reference to FIG. 4, one alternative embodiment of a stent delivery catheter is shown. Delivery catheter 400 preferably includes an elongate, flexible tubular shaft 404, having a proximal end 406 and a distal end 408. The shaft 404 defines one or more passages or lumens extending through the shaft.

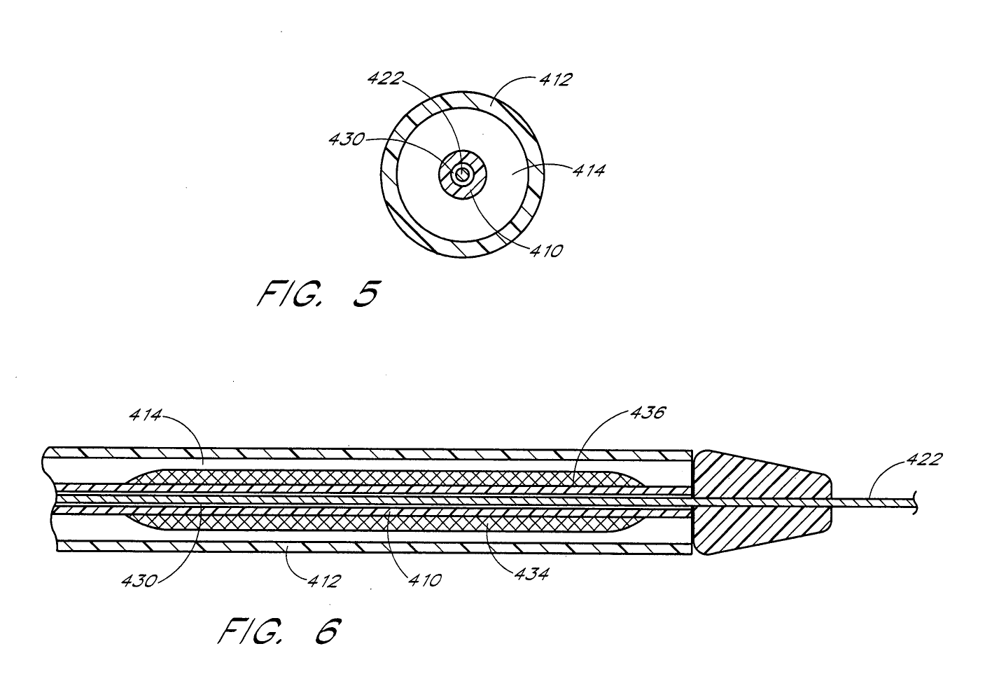

An inner member 410 and an outer member 412 are preferably arranged in coaxial alignment, as shown in FIG. 5. Member 412 forms an inner lumen 414. Inner member 410 is slidably positioned within inner lumen 414 of outer member 412 and relative axial movement between the two members is provided by inner member control handle 424 and outer member control handle 426.

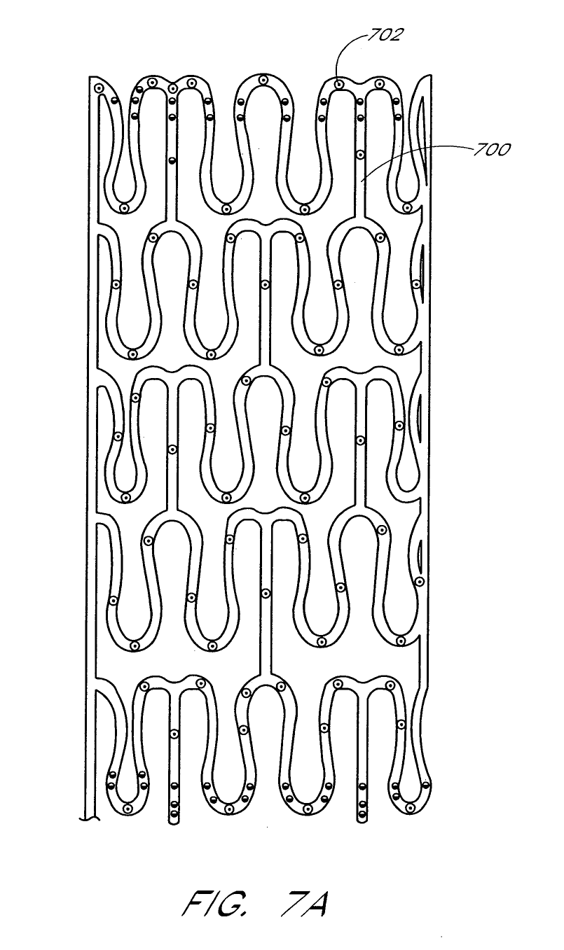

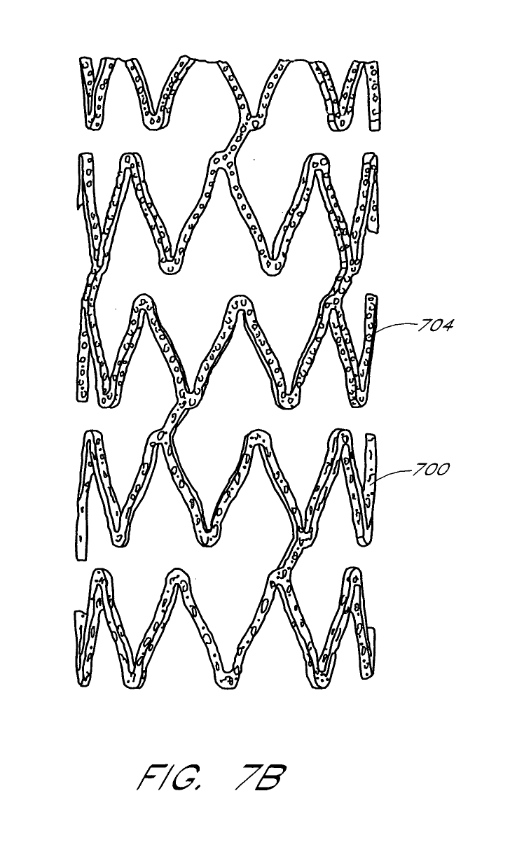

A self-expanding stent 434, as shown in FIG. 6, having an open lattice structure is mounted within the distal end 408 of catheter 400. Stent 434 comprises a tubular structure, having an inner lumen 436. Self-expanding stent 434 can take virtually any configuration that has an open lattice structure. Configurations, such as helices, coils, braids, expandable tube stents, roving wire stents, and wire mesh stents or the like may be utilized depending on the application for the device. Many stent configurations are known.

The self-expanding stent 434 is inserted in outer member inner lumen 414 and positioned at the outer member distal end. In those instances where self-expanding stent 434 is made from stainless steel or a similar material that is biased outwardly, stent 434 will be compressed and inserted into inner lumen 414. Thereafter, the distal end of inner member 410 is positioned within stent inner lumen 436 so that the outer surface of inner member 410 can come into contact with the stent inner lumen 436.

Inner member 410 is preferably made from a polymeric material that either is soft by design, or will become soft when heat is applied. The intent is to removably attach self-expanding stent 434 on the outer surface of inner member 410. Inner member 410 will partially fill the open lattice structure of stent 434 so that the stent 434 cannot move in an axial direction along the outer surface of inner member 410.

Self-expanding stent 434 is mounted on outer surface at the distal end of inner member 410. Due to the coaxial arrangement between inner member 410 and outer member 412, the inner lumen 414 of outer member 412 covers self-expanding stent 434 and helps to retain the stent on the outer surface of the inner member 410.

The size of stent 434 varies, depending on the particular treatment and access site. The overall length, diameter and wall thickness may vary based on the treatment. In a preferred embodiment, stent 434 has an inflated length between about 1 and 10 cm, preferably about 4 cm. In a preferred embodiment, stent 434 has an inflated diameter between about 0.1 and 1.5 cm. However, stents of any dimensions may be used.

A guide wire lumen 430 which preferably extends through the catheter is configured to receive guide wire 422. In order to implant self-expanding stent 434, guide wire 422 is positioned in a patient's body lumen, and typically guide wire 422 extends past a stenosed region. Distal end 408 is threaded over the proximal end of the guide wire which is outside the patient and catheter 400 is advanced along the guide wire until distal end 408 of catheter 400 is positioned within the stenosed region.

Typically, a stiffening mandrill may be incorporated in the proximal region of catheter 400 to enhance the pushability of the catheter through the patient's vascular system, and to improve the trackability of the catheter over the guide wire.

Catheters 100, 400 are used to implant the stent in a body lumen using an over-the-wire or rapid-exchange catheter configuration. Over-the-wire catheters are known in the art and details of the construction and use are set forth in U.S. Pat. Nos. 5,242,399, 4,468,224, and 4,545,390, which are herein incorporated by reference. Rapid-exchange catheters are also known in the art and details of the construction and use are set forth in U.S. Pat. Nos. 5,458,613; 5,346,505; and 5,300,085, which are incorporated herein by reference.

Catheter manufacturing techniques are generally known in the art, including extrusion and coextrusion, coating, adhesives, and molding. The disclosed catheter is preferably made in a conventional manner. The elongate shaft of the catheter is preferably extruded. The elongate shaft is preferably made of a polymer such as Nylon, the stiffness of which may be selected as appropriate. Material selection varies based on the desired characteristics. The joints are preferably bonded. Biocompatible adhesives are preferably used to bond the joints. The balloon is also preferably made in a conventional manner. However, other configurations are also acceptable.

FIGS. 7A-7H show different preferred embodiments of the stent of the present invention. A number of different types of stents including balloon-expanding, self-expanding, tubular graft stents and any other type of stent may be used.

Balloon-expanding stents, as shown in FIGS. 7A-7E, such as the well-known Palmaz-Schatz balloon expandable stent, are designed to be expanded and deployed by expanding a balloon. Various kinds and types of stents are available in the market, and many different currently available stents are acceptable for use in the present invention, as well as new stents which may be developed in the future. The stent 700 depicted in the drawings is a cylindrical metal mesh stent having an initial crimped outer diameter, which may be forcibly expanded by the balloon to a deployed diameter. When deployed in a body passageway of a patient, the stent may be designed to preferably press radially outward to hold the passageway open. The stents 700 are preferably formed from a stainless steel material. These stents are representative of a large number of stents which can be adapted for use.

Any balloon expandable stent may be used. Many are known in the art including plastic and metal stents. Some are more well known such as the stainless steel stent shown in U.S. Pat. No. 4,735,665; the wire stent shown in U.S. Pat. No. 4,950,227; another metal stent shown in European Patent Application EP0 707 837 A1 and that shown in U.S. Pat. No. 5,445,646, or 5,242,451, the disclosures of which are incorporated herein by reference.

Self-expanding stents, as shown in FIGS. 7A-7E, such as the well-known Wallstent Endoprosthesis, as described in U.S. Pat. No. 4,655,771 to Wallsten, incorporated herein by reference, expand from a contracted condition where they are mounted on the catheter assembly, to an expanded condition where the stent 700 comes in contact with the body lumen. The stents are self-expanding, which can be achieved by several means. The stents 700 are preferably formed from a stainless steel material and are configured so that they are biased radially outwardly and they will expand outwardly unless restrained. The stents 700 also can be formed from a heat sensitive material, such as nickel titanium, which will self-expand radially outwardly upon application of a transformation temperature. These stents are representative of a large number of stents which can be adapted for use with the present invention.

Tubular graft stents, as shown in FIGS. 7F-7G, include a tubular graft 712, 714 attached to a stent 700. The tubular graft 712, 714 may be a biocompatible porous or nonporous tubular structure to which a stent structure 700, such as a wire mesh, may be attached. The stent structure 700 may be biased to assume an enlarged configuration corresponding to a target treatment site, but may be constrained in a contracted condition to facilitate introduction into a patient's vasculature. The tubular graft 712, 714 preferably a peripheral wall defining a periphery and a lumen therein, the lumen extending between the first and second ends of the tubular graft. The tubular graft may be provided from a polymeric material, such as polyester, polytetrafluorethaline, Dacron, Teflon, and polyurethane. The stent may be attached to the tubular graft by sutures, staples, wires, or an adhesive, or alternatively by thermal bonding, chemical bonding, and ultrasonic bonding. The stent is preferably formed from a metallic material, such as stainless steel or Nitinol, and may be a flat-coiled sheet with one or more serpentine elements formed therein, or a wire formed into a serpentine shape. The stent 700 may be attached to an exterior surface of the tubular graft, to an interior surface of the tubular graft, or embedded in the wall of the tubular graft. The stent 700 preferably is provided along the entire length of the graft 712, as shown in FIG. 7F. However, it is also envisioned that the stent may extend over a portion of the tubular graft. Alternatively, the graft 714 may cover only a portion of the stent 700, as shown in FIG. 7G.

Configurations, such as helices, coils, braids, expandable tube stents, roving wire stents, and wire mesh stents or the like may be utilized with any of the above-described stents depending on the application for the device.

The stents as described herein can be formed from any number of materials, including metals, metal alloys and polymeric materials. Preferably, the stents are formed from metal alloys such as stainless steel, tantalum, or the so-called heat sensitive metal alloys such as nickel titanium (NiTi). The stent may be made of any suitable biocompatible material such as a metallic material or an alloy, examples of which include, but are not limited to, stainless steel, elastinite (Nitinol), tantalum, nickel-titanium alloy, platinum-iriidium alloy, gold, magnesium, or combinations thereof. Alloys of cobalt, nickel, chromium, and molybdenum may also be used. The stents may also be made from bioabsorbable or biostable polymers. Stents formed from stainless steel or similar alloys typically are designed, such as in a helical coil or the like, so that they are spring biased outwardly.

With respect to stents formed from shape-memory alloys such as NiTi (nickel-titanium alloy), the stent will remain passive in its martensitic state when it is kept at a temperature below the transition temperature. In this case, the transition temperature will be below normal body temperature, or about 98.6° F. When the NiTi stent is exposed to normal body temperature, it will immediately attempt to return to its austenitic state, and will rapidly expand radially outwardly to achieve its preformed state. Details relating to the properties of devices made from nickel-titanium can be found in “Shape-Memory Alloys,” Scientific American, Vol. 281, pages 74-82 (November 1979), which is incorporated herein by reference.

The pattern of the stent can be cut from either a cylindrical tube of the stent material or from a flat piece of the stent material, which is then rolled and joined to form the stent. Methods of cutting the lattice pattern into the stent material include laser cutting and chemical etching, as described in U.S. Pat. No. 5,759,192 issued to Saunders and U.S. Pat. No. 5,421,955 issued to Lau, both patents incorporated herein by reference in their entirety. Alternative embodiments, as known to those of skill in the art, of manufacturing stents may also be used. The stents may also be polished, as known to those of skill of the art.

In a preferred embodiment, the stents of the present invention are used to deliver more than one drug to a desired body location. Thus, treatment for different causes may be administered with a combination of drugs. In addition, more than one drug may be used for the same cause of restenosis, such that a reduced dosage may be administered, with lower risk of side-effects, and/or a more effective treatment of the cause. In addition, more than one drug may be administered for multiple causes of restenosis. Both long term therapies and short term therapies may be utilized. As used in this application, the term “drug” denotes any compound which has a desired pharmacological effect, or which is used for diagnostic purposes. Useful drugs include, but are not limited to angiogenic drugs, smooth muscle cell inhibitors, collagen inhibitors, vasodilators, anti-platelet substances, anti-thrombotic substances, anti-coagulants, gene therapies, cholesterol reducing agents and combinations thereof. The drugs may also include, but are not limited to anti-inflammatory, anti-proliferative, anti-allergic, calcium antagonists, thromboxane inhibitors, prostacyclin mimetics, platelet membrane receptor blockers, thrombin inhibitors and angiotensin converting enzyme inhibitors, antineoplastic, antimitotic, antifibrin, antibiotic, and antioxidant substances as well as combinations thereof, and the like.

Examples of these drugs include heparin, a heparin derivative or analog, heparin fragments, colchicine, agiotensin converting enzyme inhibitors, aspirin, goat-anti-rabbit PDGF antibody, terbinafine, trapidil, interferongamma, steroids, ionizing radiation, fusion tonixins, antisense oligonucleotides, gene vectors (and other gene therapies), rapamycin, cortisone, taxol, carbide, and any other such drug. Examples of such antineoplastics and/or antimitotics include paclitaxel, docetaxel, methotrexate, azathioprine, vincristine, vinblastine, fluorouracil, doxorubicin hydrochloride, and mitomycin. Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax. Examples of such cytostatic or antiproliferative agents include angiopeptin, angiotensin converting enzyme inhibitors such as captopril, cilazapril or lisinopril; calcium channel blockers (such as nifedipine), colchicine, fibroblast growth factor (FGF) antagonists, fish oil (omega 3-fatty acid), histamine antagonists, lovastatin (an inhibitor of antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax. Examples of such cytostatic or antiproliferative agents include angiopeptin, angiotensin converting enzyme inhibitors such as captopril, cilazapril or lisinopril; calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug), monoclonal antibodies (such as those specific for Platelet-Derived Growth Factor (PDGF) receptors), nitroprusside, phosphodiesterase inhibitors, prostaglandin inhibitors, suramin, seratonin blockers, steroids, thioprotease inhibitors, triazolopyrimidine (a PDGF antagonist), and nitric oxide. An example of an antiallergenic agent is permirolast potassium. Other therapeutic substances or agents that may be used include alpha-interferon, genetically engineered epithelial cells, and dexamethasone. In other examples, the therapeutic substance is a radioactive isotope for prosthesis usage in radiotherapeutic procedures. Examples of radioactive isotopes include, but are not limited to, phosphoric acid, palladium, cesium, and iodine. While the preventative and treatment properties of the foregoing therapeutic substances or agents are well-known to those of ordinary skill in the art, the substances or agents are provided by way of example and are not meant to be limiting. Other therapeutic substances are equally applicable.

The therapeutic agent may also be provided with a pharmaceutically acceptable carrier and, optionally, additional ingredients such as antioxidants, stabilizing agents, permeation enhancers, and the like. The drugs may also include radiochemicals to irradiate and/or prohibit tissue growth or to permit diagnostic imaging of a site.

Pits, pores, grooves, coatings, impregnateable materials, or a combination of these may be used to provide the drugs on the stent. In addition, a stent may include reservoirs or micropores to deliver drugs to the treatment site. Alternatively, the stent may include protruding structures which may have a central depression which may contain a therapeutic substance. Protruding structures are disclosed in U.S. Pat. No. 6,254,632, the disclosure of which is hereby incorporated by reference. These pits, pores, grooves, reservoirs, and protruding structures may be of any shape and size which may permit adequate drug delivery to the treatment site.

FIGS. 7A-7E show several embodiments of stents, as previously discussed. FIGS. 7A-7E also show pits, pores, or spheres, 702 (FIG. 7A), 704 (FIG. 7B), 706 (FIG. 7C); and reservoirs, 708 (FIG. 7D), 710 (FIG. 7E). FIG. 7H shows pores 716 and reservoirs 718 in detail, which may be used in combination, as shown.

In an alternative embodiment, the stent may comprise a plurality of microencapsulated spheres containing a medicament, the microencapsulated spheres being disposed about the exterior surface of the stent so as to rupture upon radial expansion of the stent by a predetermined amount. The microencapsulated spheres are preferably encapsulated in a coating applied to the exterior surface of the stent. The spheres are preferably made from a bioabsorbable or biostable material.

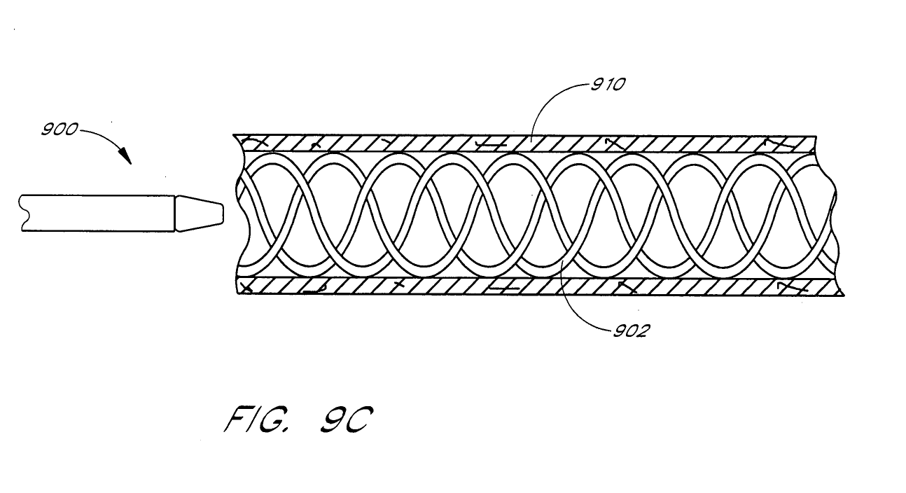

FIG. 71 shows a stent 700 having a coating 720. Applying a coating to the metal, attaching a covering or membrane, or embedding material on the surface via ion bombardment may be used to apply the drugs. Conventionally, drugs are incorporated into a polymer material which is then coated on the stent. The coating material should be able to adhere strongly to the metal stent both before and after expansion, be capable of retaining the drug at a sufficient load level to obtain the required dose, be able to release the drug in a controlled way over a period of time, and be as thin as possible so as to minimize the increase in profile. In addition, the coating material should not contribute to any adverse response by the body. A coating may be located on the interior or exterior surfaces, or both surfaces, of the stent. In a preferred embodiment, multiple coatings may be provided with the stent. Each coating preferably comprises a different drug.

In an alternative embodiment, as shown in FIG. 7J, a drug-impregnated film 722 is provided in the open spaces 724 of the stent. The film may completely surround the stent, or the film may alternatively cover only one, two, or more of the spaces 724 between the individual stent struts. The film 722 is shown with cross-hatching in FIG. 7J. The cross-hatching does not indicate that the film is necessarily porous, but merely indicates the presence of the film 722; however, it is envisioned that the film may be porous. The film is preferably non-porous.

Preferably, the drug delivery film dissolves and is absorbed by the body, releasing the drug at the treatment site. The film provides uniform drug delivery to the body lumen being treated. Accordingly, lower dosages of drugs are generally required to treat the site.

The film is preferably attached to the stent by an adhesive, or alternatively by thermal bonding, chemical bonding, and/or ultrasonic bonding. Alternatively, the film is formed on the stent by depositing the film material onto a balloon and stent assembly. As with stent coatings, the film material should be able to adhere strongly to the metal stent both before and after expansion, be capable of retaining the drug at a sufficient load level to obtain the required dose, be able to release the drug in a controlled way over a period of time, and be as thin as possible. In addition, the film material should not contribute to any adverse response by the body.

Alternatively, or in addition to a coating and/or film, the stent may contain reservoirs which can be loaded with the drugs. A coating or membrane of biocompatible material could be applied over the reservoirs which would control the diffusion of the drug from the reservoirs to the body lumen. The size, shape, position and number of reservoirs can be used to control the amount of drug, and therefore the dose delivered.

The reservoirs or pores are typically covered by a polymeric layer. Polymeric materials that can be used for the layer are typically either bioabsorbable or biostable. A bioabsorbable polymer bio-degrades or breaks down in the body and is not present sufficiently long after implantation to cause an adverse local response. Bioabsorbable polymers are gradually absorbed or eliminated by the body by hydrolysis, metabolic process, bulk, or surface erosion. Examples of bioabsorbable, biodegradable materials include but are not limited to polycaprolactone (PCL), poly-D, L-lactic acid (DL-PLA), poly-L-lactic acid (L-PLA), poly(lactide-co-glycolide), poly(hydroxybutyrate), poly(hydroxybutyrate-co-valerate), polydioxanone, polyorthoester, polyanhydride, poly(glycolic acid), poly(glycolic acid cotrimethylene carbonate), polyphosphoester, polyphosphoester urethane, poly (amino acids), cyanoacrylates, poly(trimethylene carbonate), poly(iminocarbonate), copoly(ether-esters), polyalkylene oxalates, polyphosphazenes, polyiminocarbonates, and aliphatic polycarbonates. Biomolecules such as heparin, fibrin, fibrinogen, cellulose, starch, and collagen are typically also suitable. Examples of biostable polymers include Parylene, Parylast, polyurethane (for example, segmented polyurethanes), polyethylene, polyethylene teraphthalate, ethylene vinyl acetate, silicone and polyethylene oxide.

The stent may be impregnated with two or more drugs by any known process in the art including high pressure loading in which the stent is placed in a bath of the desired drug or drugs and subjected to high pressure or, alternatively, subjected to a vacuum. The drug may be carried in a volatile or non-volatile solution. In the case of a volatile solution, following loading of the drug, the volatile carrier solution may be volatilized. In the case of the vacuum, the air in the pores of the metal stent is evacuated and replaced by the drug-containing solution.

In accordance with the present invention, the stent may further be coated with multiple layers of one or more therapeutic agents to allow for longer term drug elution, preferably employing a number of different drugs over time. As such, the drug in the pores would not be eluted until the coating of drug has been absorbed, thereby allowing for longer term drug treatment than would be available from the coated metal alone.

One agent is preferably a quick-release drug for immediate treatment of the body lumen, while another agent is a slow-release drug for long-term treatment of the body lumen. Preferably, these drugs, whether slow-release or quick-release, are provided in low dosages. Accordingly, lower dosages are used to treat the site, improving long-term therapy and reducing the risk of side-effects due to the therapeutic agents. In a preferred embodiment, two, three, four or more drugs, preferably in low dosages, are used in combination. Advantageously, the dosage is selected such that the risk of side-effects from the combination of low-dosage therapeutic agents is reduced in comparison to the risk of side-effects from a conventional dosage of either drug administered at conventional dosage levels.

Preferably, the therapeutic agents are delivered simultaneously. For example, an anti-inflammatory and anti-thrombogenic drug may be required at the same time. An anti-inflammatory is generally required during the initial implantation of the stent, where as an anti-thrombogenic is generally required during the entire time the stent is implanted in the body. Accordingly, at least two drugs are preferably provided simultaneously.

In addition, the drugs are preferably administered immediately upon stent deployment in the body lumen. Restenosis occurs immediately upon deployment of a stent. Accordingly, the drugs should also be delivered immediately.

As the stent coatings or microspheres biodegrade, drugs are administered to the surrounding tissue or to the blood stream. The rate of drug release is preferably controlled by the rate of degradation of the biodegradable materials. Accordingly, a material that degrades rapidly will release the agent faster, while a material that degrades slower will release the agent slower. Additionally, the rate of drug release can either accelerate or slow down the rate of degradation of the biodegradable material. Thus, the rate of release of a drug may act as a control quantity for the rate of degradation.

In a preferred embodiment, release of the therapeutic agents from the stent may also be stimulated by a variety of methods, including electrical or mechanical stimulation, such as heat or ultrasound energy.

In an alternative embodiment, the balloon may also be coated with a drug. Typically, a hydrogel coating may be used in combination with a balloon. A hydrogel is a cross-linked polymer material formed from the combination of a colloid and water. The drug is held within the hydrogen-bond matrix formed by the gel.

Additional pores or reservoirs may be manufactured into the stent, into which drugs are incorporated. These pores or reservoirs may be manufactured using chemical etching or laser techniques, as previously discussed herein, or by other means known.

Some techniques for incorporating drugs include simple mixing or solubilizing with polymer solutions, dispersing into the biodegradable polymer during the extrusion of melt spinning process, or coating onto an already formed stent. In one embodiment, hollow fibers, which contain anti-thrombogenic drugs, are arranged in a parallel concentric configuration with solid fibers for added support for use on the outer surface of the stent.

Further, drugs can be incorporated into the coating(s) of both the inner and/or outer surfaces by using methods such as melting or solvation. If an interior film layer is present within the main body as well, the interior layer and inner and outer surfaces are then combined with each other such as by mechanically pressing one layer to the other layer in a process augmented by heat or solvation adhesives. In another embodiment, drugs or biologically active agents are incorporated into the film layer and surfaces by entrapment between the layers and surfaces of biodegradable material sandwiched together, thereby further promoting release of the drugs or agents at different rates.

A variety of methods may be used to apply a coating to a stent including vapor deposition, spray coating, and ion beam assisted deposition. In addition, in a preferred embodiment, by exposing a coated device to a low energy, relatively non-penetrating energy source such as gas plasma, electron beam energy, or corona discharge, the coating is stabilized to permit timed or long-term delivery of the drug.

Although a number of methods for applying drugs to a stent have been discussed, additional methods of incorporating drugs with a stent are known in the art and may be used.

In a preferred embodiment, the patient is tested for allergies to the drugs and/or stent material(s) prior to implantation of the drug delivery stent.

FIG. 7K shows an alternative embodiment of a stent. Stent 700 includes a plurality of barbs 726 at both ends 728. It is known in the art that the body lumen tends to collapse at the ends of a stent. Stent 700 provides additional support at the stent ends 728 to prevent or reduce the collapse of the body lumen near the ends. The stent is shown having two barbs 726 at either end 728; however, it is envisioned that more than two barbs may be provided at each end. The barbs 726 provide a transition between the region of the vessel which is entirely supported by the stent 700 and a region which is not supported by the stent.

With reference to FIGS. 8A-C and 9A-C, the method of delivering a stent of the present invention is shown. As previously discussed self-expanding and balloon expanding stents may be used. A delivery system for balloon expanding stents, and a delivery system for self-expanding stents have also been described herein. Tubular graft stents may be used with either self-expanding or balloon-expanding systems.

In either system, the delivery system is preferably percutaneously delivered to the treatment site. The stent is percutaneously introduced in the contracted condition, advanced to a treatment site within a body vessel, and deployed to assume an enlarged condition and repair and/or bypass the treatment site.

A method of delivering a stent system as described above generally includes locating the site to be treated, providing a suitable delivery catheter, positioning the distal portion of a delivery catheter with a stent disposed thereon or therein in the branch of the site to be treated, partially deploying the stent in a vessel, adjusting the position of the stent if necessary, and then fully deploying the stent. Methods of navigating catheters through blood vessels or other fluid conduits within the human body are well known, and will therefore not be discussed herein.

With respect to the balloon expanding delivery system 800 as shown in FIGS. 8A-8C, a method frequently described for delivering a stent to a desired intraluminal location includes mounting the expandable stent 802 on an expandable member 804, such as a balloon, provided on the distal end 806 of a catheter 808, advancing the catheter to the desired location 810 within the patient's body lumen 812 (FIG. 8A), inflating the balloon 804 (FIG. 8B) on the catheter 800 to expand the stent 802 into a permanent expanded condition and then deflating the balloon 804 and removing the catheter 800. When fully deployed and implanted, as shown in FIG. 8C, stent 802 will support and hold open stenosed region 810 so that blood flow is not restricted.

With respect to the self-expanding delivery system 900 as shown in FIGS. 9A-9C, self-expanding stent 902 is implanted in stenosed region 904 by moving outer member 906 in a proximal direction while simultaneously moving inner member 908 in a distal direction (FIG. 9A). With reference to FIG. 9B, as portions of self-expanding stent 902 are no longer contained by outer member 906, it will expand radially outwardly into contact with vessel wall 910 in the area of stenosed region 904. When fully deployed and implanted, as shown in FIG. 9C, stent 902 will support and hold open stenosed region 904 so that blood flow is not restricted.

In order to visualize the position of a partially or fully-deployed stent with a suitable radiographic apparatus, a contrast media may be introduced through the catheter to the region of the stent placement. Many suitable contrast media are known to those skilled in the art. The contrast media may be introduced at any stage of the deployment of the stent system. For example, a contrast media may be introduced after partially deploying the stent, or after fully deploying the stent.

Under exposure to body tissue, the drug coatings or other drug delivery means are biodegraded and absorbed by the body. To maintain the pharmacological activity after delivery, additional amounts or types of drugs may be provided on additional layers, which are similarly biodegraded and absorbed by the body. In a preferred embodiment, the stent is coated or embedded with drugs such that the drugs are released at different rates. As each layer or coating is biodegraded, different types and/or quantities of drugs are released to and absorbed by the body.

Although the present invention has been described in terms of certain preferred embodiments, other embodiments of the invention including variations in dimensions, configuration and materials will be apparent to those of skill in the art in view of the disclosure herein. In addition, all features discussed in connection with any one embodiment herein can be readily adapted for use in other embodiments herein. The use of different terms or reference numerals for similar features in different embodiments does not imply differences other than those which may be expressly set forth. Accordingly, the present invention is intended to be defined solely by reference to the appended claims, and not limited to the preferred embodiments disclosed herein.

1. A stent delivery system, comprising:
an elongate shaft comprising a proximal end and a distal end and a lumen extending therein; and
a stent, at the distal end of said shaft, comprising at least two drugs, wherein a first therapeutic drug is a quick-release drug, and a second therapeutic drug is a slow-release drug.

2. The stent of claim 1, wherein said stent is balloon-expandable.

3. The stent of claim 1, wherein said stent is self-expanding.

4. The stent of claim 1, wherein said stent comprises a tubular graft.

5. The stent of claim 1, wherein said drugs are selected from the group consisting of heparin, heparin derivatives, heparin fragments, colchicine, angiopeptin, steroids, gene vectors, cortisone, taxol, nitric oxide, carbide, docetaxel, mthotrexate, azathiprine, vincristine, vinblastine, fluorouracil, doxorubicin hydrochloride, mitomycin, heparinoids, hirudin, argatroban, forskolin, vapiprost, prostacyclin, protacyclin analogues, dextran, dipryidamole, recombinant hirudin, captrpril, cilazapril, lisinopril, calcium channel blockers, fish oil, histamine antagonists, lovastatin, dipryidamole, monoclonal antibodies, suramin, seratonin blockers, thioprotease inhibitors, triazolpyrimidine, permirolast potassium, dexamethason, radioactive isotopes, phosphoric acid, palladium, cesium, iodine and aspirin.

6. The stent of claim 1, wherein said drugs are selected from the group consisting of anti-thrombotics, anti-inflammatories, anti-proliferatives, antineoplastic, antiplatelet, antifibrin, antibiotic, antioxidant, anti-allergic drugs, angiogenic drugs, smooth muscle cell inhibitors, anti-coagulents, cholesterol reducing agents, calcium antagonists, thromboxane inhibitors, prostacyclin mimetics, platelet membrane receptor blockers, thrombin inhibitors, angiotensin converting enzyme inhibitors and combinations thereof.
